(19) United States
(12) Patent Application Publication
(10) Pub. No.: US 2026/0109942 A1
(43) Pub. Date: Apr. 23, 2026

(54) CRISPR KNOCKDOWN OT ACIDITHIOBACILLUS FERROOXIDANS GENES

(71) Applicant: The Trustees of Columbia University in the City of New York, New York, NY (US)

(72) Inventors: SCOTT BANTA, Fairfield, CT (US); HEEJUNG JUNG, New York, NY (US); YUTA INABA, New York, NY (US)

(73) Assignee: The Trustees of Columbia University in the City of New York, New York, NY (US)

(21) Appl. No.: 19/376,033

(22) Filed: Oct. 31, 2025

Related U.S. Application Data
(63) Continuation of application No. PCT/US2024/027270, filed on May 1, 2024.
(60) Provisional application No. 63/463,296, filed on May 1, 2023.

(51) Int. Cl. C12N 1/20; C12N 9/22; C12N 15/113; C12N 15/74; C12R 1/01

(52) U.S. Cl. CPC C12N 1/20; C12N 9/226; C12N 15/113; C12N 15/74; C12N 2310/20; C12N 2310/531; C12R 2001/01

(57) ABSTRACT

Methods and genomic editing systems for Acidithiobacillus ferrooxidans are described herein involving use of Cpf1 (Cas12a).

### PRIORITY

This application is a continuation of and claims the benefit of International Application No. PCT/US24/27270, filed May 1, 2024, which claims the benefit of priority of U.S. provisional patent application No. 63/463,296, filed on May 1, 2023, the benefit of priority of which is claimed hereby, and which is incorporated by reference herein in its entirety.

### STATEMENT OF GOVERNMENT SUPPORT

This invention was made with government support under DE-AR0001340 awarded by the Advanced Research Projects Agency-Energy. The government has certain rights in the invention.

### INCORPORATION BY REFERENCE OF SEQUENCE LISTING

This application contains a Sequence Listing which has been submitted electronically in ST26 format and hereby incorporated by reference in its entirety. Said ST26 file, created on Apr. 28, 2024, is name 2413154WO1.xml and is 12,259 bytes in size.

### BACKGROUND

CRISPR/Cas is a microbial adaptive immune system widely found in many prokaryotes that microbes have naturally used to defend against infections (e.g., by bacteriophages). CRISPR/Cas enables multiple gene manipulations with high efficiency and as a result cas9-based editing procedures have been developed as gene-editing tools in various cell types.

Bioleaching, or biomining is a process that extracts valuable metal from low-grade ore using microorganisms such as bacteria. Acidithiobacillus ferrooxidans is an iron-oxidizing chemolithotroph that plays a key role in industrial metal bioleaching or biomining. Despite increasing scientific and industrial interest in this organism, their unusual physiological traits and growth conditions make it difficult to use this strain. Additionally, genetic engineering of A. ferrooxidans has not been successful due to low transformation and recombination efficiencies.

### SUMMARY

A gene silencing system for Acidithiobacillus ferrooxidans is described herein that includes use of Clustered Regularly Interspaced Short Palindromic Repeats (CRISPR) and CRISPR-associated gene (Cas). Acidithiobacillus ferrooxidans are one of the most important microbes involved in industrial biomining. Although Cas9 has been used in several bacterial species, recent literature has revealed that it is hard to deploy in a variety of bacteria and even has adverse effects in E. coli. For several reasons genetic engineering of Acidithiobacillus ferrooxidans cells has been difficult. For example, Acidithiobacillus ferrooxidans thrive under iron-rich (<100 mM Fe) and acidic (pH<2.0) conditions but other bacterial species do not. Hence, conjugation between Acidithiobacillus ferrooxidans and other bacterial species is inefficient, making transfer of CRISPR enzymes inefficient. The high GC content of A. ferrooxidans genomes can increase the non-specific binding of Cas9 nucleases to NGG PAM sites, further rendering Cas9 incompatible for use. An uncharacterized type IV CRISPR/Cas system has been identified in A. ferrooxidans ATCC23270, however application of this endogenous or other well-established CRISPR/Cas systems in A. ferrooxidans has proven difficult.

Methods and genomic editing systems for Acidithiobacillus ferrooxidans are described herein involving Cpf1 (Cas12a) originated from Francisella tularensis, which is a single RNA-guided endonuclease of class II CRISPR system. However, use of a modified form of Cas12a is less toxic to Acidithiobacillus ferrooxidans than Cas9 or defective forms of Cas9. In particular, a catalytically inactive form of Cas12a that can still perform genomic editing provides improved editing. Hence, a dCas12a-mediated CRISPR interference system (CRISPRi-dCas12a) can be used for editing Acidithiobacillus ferrooxidans.

One embodiment provides a method comprising introducing at least one guide RNA to at least one Acidithiobacillus ferrooxidans cell that expresses a Cas12 nuclease or modified Cas12 nuclease, to generate at least one modified Acidithiobacillus ferrooxidans cell. In one embodiment, the Cas12 nuclease or the modified Cas12 nuclease is a Cas12a nuclease or a modified Cas12a nuclease. In one embodiment, the Cas12 nuclease or the modified Cas12 nuclease is a Francisella tularensis Cas12a nuclease or a modified Francisella tularensis Cas12a nuclease. In one embodiment, the modified Cas12 nuclease has reduced catalytic activity as compared to a wild type Cas12 nuclease, wherein the modified Cas12 nuclease can perform genomic editing. In one embodiment, the modified Cas12 nuclease is a catalytically inactive form of Cas12a that can perform genomic editing. In one embodiment, the modified Cas12 nuclease has a mutation at D917 of SEQ ID NO: 1, 3 or 4 or 95% identity thereto. In one embodiment, the mutation is D917A. In another embodiment, the modified Cas12 nuclease has a mutation at E1005 of SEQ ID NO: 1, E1006 of SEQ ID NO: 3, E1006 of SEQ ID NO: 4 or 95% identity thereto. In one embodiment, the mutation is E1005A or E1006A. In one embodiment, the modified Cas12 nuclease has a D917A mutation and an E1006A or E1005A mutation. In one embodiment, at least one of the guide RNAs comprises one or more targeting sequences. In one embodiment, at least one of the guide RNAs comprises two or more targeting sequences. In one embodiment, at least one of the guide RNAs include both targeting guide RNA (crRNA) sequences and nuclease-binding guide RNA (tracrRNA) sequences in a single RNA molecule. In another embodiment, at least one of the guide RNAs target genomic sites near TTTV PAM sites in the Acidithiobacillus ferrooxidans genome. In one embodiment, at least one of the guide RNAs comprises up to two, three or four stem structures, each comprising double-stranded regions of about 3-5 nucleotides. In one embodiment, at least one of the guide RNAs comprises one or two spacers. In one embodiment, at least one of the guide RNAs are introduced to at least one Acidithiobacillus ferrooxidans cell as an expression cassette or expression vector comprising a promoter operably linked to a segment encoding at least one of the guide RNAs. One embodiment comprises Acidithiobacillus ferrooxidans with a knock down of dPetB2.

One embodiment provides a system comprising (a) an expression cassette or expression vector comprising a promoter operably linked to restriction site adapted to receive a nucleotide segment encoding at least one guide RNA; and (b) a population of Acidithiobacillus ferrooxidans cells modified to express a Cas12 nuclease or a modified Cas12 nuclease. In one embodiment, the Cas12 nuclease or the modified Cas12 nuclease is a Cas12a nuclease or a modified Cas12a nuclease. In one embodiment, the Cas12 nuclease or the modified Cas12 nuclease is a Francisella tularensis Cas12a nuclease or a modified Francisella tularensis Cas12a nuclease. In one embodiment, the modified Cas12 nuclease has reduced catalytic activity as compared to a wild type Cas12 nuclease, wherein the modified Cas12 nuclease can perform genomic editing. In one embodiment, the modified Cas12 nuclease is a catalytically inactive form of Cas12a that can perform genomic editing. In one embodiment, the modified Cas12 nuclease has a mutation at D917 of SEQ ID NO: 1, 3 or 4 or 95% identity thereto. In one embodiment, the mutation is D917A. In another embodiment, the modified Cas12 nuclease has a mutation at E1005 of SEQ ID NO: 1, E1006 of SEQ ID NO: 3, E1006 of SEQ ID NO: 4 or 95% identity thereto. In one embodiment, the mutation is E1005A or E1006A. In one embodiment, the modified Cas12 nuclease has a D917A mutation and an E1006A or E1005A mutation. In one embodiment, at least one of the guide RNAs comprises one or more targeting sequences. In one embodiment, at least one of the guide RNAs comprises two or more targeting sequences. In one embodiment, at least one of the guide RNAs include both targeting guide RNA (crRNA) sequences and nuclease-binding guide RNA (tracrRNA) sequences in a single RNA molecule. In one embodiment, at least one of the guide RNAs target genomic sites near TTTV PAM sites in the Acidithiobacillus ferrooxidans genome. In one embodiment, at least one of the guide RNAs comprises up to two, three or four stem structures, each comprising double-stranded regions of about 3-5 nucleotides. In one embodiment, at least one of the guide RNAs comprises one or two spacers.

### BRIEF DESCRIPTION OF THE FIGURES

Embodiments of the invention are described below with reference to the following accompanying drawings.

FIG. 1: gRNA structures having 1 spacer (51E-lacY2) or 2 spacers (51E-lacY3) targeted to single genes.

FIG. 2: Growth profiles of E. coli BL21 with different ddFnCas12a plasmids in media containing 0.4% lactose as the carbon source, where growth was measured as optical density at 600 nm.

FIGS. 3A-3B: Iron oxidation profiles of wild-type (WT) and knocking down (dPetB2) strains under high (5 g/L) (FIG. 3A) and low (1 g/L) (FIG. 3B) sulfur conditions.

FIG. 4: Expression of petBII transcripts in wild-type (WT) and knock down (ApetB2) A. ferrooxidans strains grown in iron and sulfur growth media with citric acid (F2S) or without citric acid (AFM1 S), in relation to 16S rRNA gene (rrs).

### DETAILED DESCRIPTION

Provided herein are compositions and methods for a gene silencing system for Acidithiobacillus ferrooxidans using Clustered Regularly Interspaced Short Palindromic Repeats (CRISPR) and CRISPR-associated gene (Cas). CRISPR/Cas is a microbial adaptive immune system which is used to defend against foreign infections and widely found in many prokaryotes. As CRISPR/Cas enables multiple gene manipulations with high efficiency and ease, it is considered as a powerful gene-editing tool in many cells.

### Definitions

References in the specification to “one embodiment,” “an embodiment,” etc., indicate that the embodiment described may include a particular aspect, feature, structure, moiety, or characteristic, but not every embodiment necessarily includes that aspect, feature, structure, moiety, or characteristic. Moreover, such phrases may, but do not necessarily, refer to the same embodiment referred to in other portions of the specification. Further, when a particular aspect, feature, structure, moiety, or characteristic is described in connection with an embodiment, it is within the knowledge of one skilled in the art to affect or connect such aspect, feature, structure, moiety, or characteristic with other embodiments, whether or not explicitly described.

The singular forms “a,” “an,” and “the” include plural reference unless the context clearly dictates otherwise. Thus, for example, a reference to “a compound” includes a plurality of such compounds, so that a compound X includes a plurality of compounds X. It is further noted that the claims may be drafted to exclude any optional element. As such, this statement is intended to serve as antecedent basis for the use of exclusive terminology, such as “solely,” “only,” and the like, in connection with any element described herein, and/or the recitation of claim elements or use of “negative” limitations.

The term “and/or” means any one of the items, any combination of the items, or all of the items with which this term is associated. The phrase “one or more” is readily understood by one of skill in the art, particularly when read in context of its usage. For example, one or more substituents on a phenyl ring refers to one to five, or one to four, for example if the phenyl ring is di-substituted.

As used herein, “or” should be understood to have the same meaning as “and/or” as defined above. For example, when separating a listing of items, “and/or” or “or” shall be interpreted as being inclusive, e.g., the inclusion of at least one, but also including more than one of a number of items, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, the terms “including,” “includes,” “having,” “has,” “with,” or variants thereof, are intended to be inclusive similar to the term “comprising.”

The term “about” can refer to a variation of ±5%, ±10%, ±20%, or ±25% of the value specified. For example, “about 50” percent can in some embodiments carry a variation from 45 to 55 percent. For integer ranges, the term “about” can include one or two integers greater than and/or less than a recited integer at each end of the range. Unless indicated otherwise herein, the term “about” is intended to include values, e.g., weight percentages, proximate to the recited range that are equivalent in terms of the functionality of the individual ingredient, the composition, or the embodiment. The term about can also modify the endpoints of a recited range as discuss above in this paragraph.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible sub-ranges as well as individual numerical values within that range. For example, description of a range such as from 1 to 6 should be considered to have specifically disclosed sub-ranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The term “expression” includes any step involved in the production of a polypeptide including, but not limited to, transcription, post-transcriptional modification, translation, post-translational modification, and secretion.

The term “expression vector” means a linear or circular DNA molecule that comprises a polynucleotide encoding a polypeptide and is operably linked to control sequences that provide for its expression.

The term “operably linked” means a configuration in which a control sequence is placed at an appropriate position relative to the coding sequence of a polynucleotide such that the control sequence directs expression of the coding sequence.

Acidithiobacillus ferrooxidans

Described herein are methods and editing systems for genetic modification of Acidithiobacillus ferrooxidans (available for example from the American Type culture Collection as strain ATCC 19859, ATCC 23270, or strain ATCC 33020). As illustrated herein, use of Cas12a or a modified Cas12a enzyme that is less active provides improved genomic editing of Acidithiobacillus ferrooxidans.

### CRISPR/Cas

The iron- and/or sulfur-oxidizing Acidithiobacillus ferrooxidans is a well-studied acidophile which plays a role in industrial metal bioleaching or biomining. Despite increasing scientific and industrial interest in this organism, their unusual physiological traits have hampered the development of genetic tools applicable to this organism. Although an uncharacterized type IV CRISPR/Cas system has been identified in A. ferrooxidans ATCC23270, application of this endogenous or other well-established CRISPR/Cas systems in A. ferrooxidans has been difficult.

Provided herein is the development of a vector carrying Cpf1 (Cas12a, a subtype of Cas12 proteins) originated from Francisella tularensis, which is a single RNA-guided endonuclease of class II CRISPR system (Cas12a can also originate from Francisella novicida, Acidaminococcus sp., Lachnospiraceae sp., Prevotella sp.). Although Cas9 has been used in several bacteria, recent literature has revealed that it is hard to deploy in a variety of bacteria and even has adverse effects in E. coli. The high GC content of A. ferrooxidans genomes likely increases non-specific binding of NGG PAM site, further rendering Cas9 incompatible for use. Thus, Cas12 is becoming an alternative to Cas9 with advantages of having its own RNA processing capability, and thereby allowing simpler guide RNA (gRNA) sequences to be used.

There are several additional reasons why Cas12a or modified forms of Cas12a are particularly useful for editing Acidithiobacillus ferrooxidans. Cas9 requires additional 80-nucleotide-long tracrRNA (trans-activating CRISPR RNA) to process crRNA (CRISPR RNA), whereas Cas12a only needs a 20-nucleotide direct repeat sequence preceding the crRNA. Simpler guide RNA (gRNA) sequences can therefore be used with Cas12a nucleases. Because Cas12a can process maturation of crRNA arrays itself, use of Cas12a systems do not require other RNase activities. Such self-processing of crRNA arrays by Cas12a facilitates multiplexed genome editing at high efficiencies.

CRISPR/Cas systems include protospacer adjacent motifs (PAM) that are short DNA sequences (usually 2-6 base pairs in length) near the DNA region targeted for cleavage by a CRISPR system. Cas9 recognizes a G-rich PAM sequence (5′-NGG, where N can be any of four nucleotides). On the other hand, Cas12a binds T-rich PAM sites (5′TTTN). The unique PAM sequences of Cas12a systems significantly increase the on-target editing efficiency of Cas12a in high GC-content Acidithiobacillus ferrooxidans genomes (compared to Cas9 systems), due to the lower chance of the Cas12a system misreading the PAM sequences in the high GC genome.

Following recognition of a PAM site, Cas9 cleaves target DNA upstream sequence at proximal position of the PAM, while the DNA repair system (for genome editing) usually destroys the PAM site because of the close proximity. This prevents future genome editing and Cas9 re-targeting. However, Cas12a cleaves the target DNA 18-23 nucleotides downstream of the PAM site. Thus, when using Cas12a editing systems, the PAM sites will remain available after DNA editing, which enables repeated cleavage events and thus improving the on-target editing efficiency.

A further drawback of Cas9 is its toxicity in different bacteria.

The editing compositions and methods described herein involve use of Cas12 as an alternative to other Cas nucleases, including Cas9.

For example, a sequence of a Francisella tularensis subsp. novicida FTG type V CRISPR-associated protein Cas12a/Cpf1 is shown below (NCBINZ_DS995364.1; SEQ ID NO:1).

| | |
|---|---|
| 1 | MSIYQEFVNK YSLSKTLRFE LIPQGKTLEN IKARGLILDD |
| 41 | EKRAKDYKKA KQIIDKYHQF FIEEILSSVC ISEDLLQNYS |
| 81 | DVYFKLKKSD DDNLQKDFKS AKDTIKKQIS KYINDSEKFK |
| 121 | NLFNQNLIDA KKGQESDLIL WLKQSKDNGI ELFKANSDIT |
| 161 | DIDEALEIIK SFKGWITYFK GFHENRKNVY SSNDIPTSII |
| 201 | YRIVDDNLPK FLENKAKYES LKDKAPEAIN YEQIKKDLAE |
| 241 | ELTFDIDYKT SEVNQRVFSL DEVFEIANFN NYLNQSGITK |
| 281 | FNTIIGGKFV NGENTKRKGI NEYINLYSQQ INDKTLKKYK |
| 321 | MSVLFKQILS DTESKSFVID KLEDDSDVVT TMQSFYEQIA |
| 361 | AFKTVEEKSI KETLSLLFDD LKAQKLDLSK IYFKNDKSLT |
| 401 | DLSQQVFDDY SVIGTAVLEY ITQQVAPKNL DNPSKKEQDL |
| 441 | IAKKTEKAKY LSLETIKLAL EEFNKHRDID KQCRFEEILS |
| 481 | NFAAIPMIFD EIAQNKDNLA QISIKYQNQG KKDLLQASAE |
| 521 | EDVKAIKDLL DQTNNLLHRL KIFHISQSED KANILDKDEH |
| 561 | FYLVFEECYF ELANIVPLYN KIRNYITQKP YSDEKFKLNF |
| 601 | ENSTLASGWD KNKESANTAI LFIKDDKYYL GIMDKKHNKI |
| 641 | FSDKAIEENK GEGYKKIVYK QIADASKDIQ NLMIIDGKTV |
| 681 | CKKGRKDRNG VNRQLLSLKR KHLPENIYRI KETKSYLKNE |
| 721 | ARFSRKDLYD FIDYYKDRLD YYDFEFELKP SNEYSDFNDF |
| 761 | TNHIGSQGYK LTFENISQDY INSLVNEGKL YLFQIYSKDF |
| 801 | SAYSKGRPNL HTLYWKALFD ERNLQDVVYK LNGEAELFYR |
| 841 | KQSIPKKITH PAKETIANKN KDNPKKESVF EYDLIKDKRF |
| 881 | TEDKFFFHCP ITINFKSSGA NKFNDEINLL LKEKANDVHI |
| 921 | LSIDRGERHL AYYTLVDGKG NIIKQDNFNI IGNDRMKTNY |
| 961 | HDKLAAIEKD RDSARKDWKK INNIKEMKEG YLSQVVHEIA |
| 1001 | KLVIEYNAIV VFEDLNFGFK RGRFKVEKQV YQKLEKMLIE |
| 1041 | KLNYLVFKDN EFDKTGGVLR AYQLTAPFET FKKMGKQTGI |
| 1081 | IYYVPAGFTS KICPVTGFVN QLYPKYESVS KSQEFFSKFD |
| 1121 | KICYNLDKGY FEFSFDYKNF GDKAAKGKWT IASFGSRLIN |
| 1061 | FRNSDKNHNW DTREVYPTKE LEKLLKDYSI EYGHGECIKA |
| 1201 | AICGESDKKF FAKLTSVLNT ILQMRNSKTG TELDYLISPV |
| 1241 | ADVNGNFFDS RQAPKNMPQD ADANGAYHIG LKGLMLLDRI |
| 1281 | KNNQEGKKLN LVIKNEEYFE FVQNRNN |

The D917 and/or E1005 residues of the SEQ ID NO:1 protein (highlighted above) can be mutated to generate a catalytically inactive form of this Cas12a (ddFnCas12a) protein. Any substitution at D917 and/or E1005 residue of SEQ ID NO:1 (or similar mutations/substitutions in SEQ ID NO: 3 and/or 4) that results in a protein with reduced (including about 5%, about 10%, about 15%, 20, about 25%, about 30%, about 35%, about 40%, about 45%, about 50%, about 55%, about 60%, about 65%, about 70%, about 75%, about 80%, about 85%, about 90%, about 95% reduction in activity as compared to wild-type) or no catalytically active protein, as compared to the non-mutated sequence (e.g., wild type) can be used in the methods provided herein. For example, the D917A/E1005A double mutation can be used as the ddFnCas12a catalytically inactive protein.

A nucleotide sequence for the SEQ ID NO:1 Francisella tularensis Cas12a/Cpf1 protein is shown below (NCBI NZ DS995364.1; SEQ ID NO:2).

| | |
|---|---|
| 1 | ATGTCAATTT ATCAAGAATT TGTTAATAAA TATAGTTTAA |
| 41 | GTAAAACTCT AAGATTTGAG TTAATCCCAC AGGGTAAAAC |
| 81 | ACTTGAAAAC ATAAAAGCAA GAGGTTTGAT TTTAGATGAT |
| 121 | GAGAAAAGAG CTAAAGACTA CAAAAAGGCT AAACAAATAA |
| 161 | TTGATAAATA TCATCAGTTT TTTATAGAGG AGATATTAAG |
| 201 | TTCGGTTTGT ATTAGCGAAG ATTTATTACA AAACTATTCT |
| 241 | GATGTTTATT TTAAACTTAA AAAGAGTGAT GATGATAATC |
| 281 | TACAAAAAGA TTTTAAAAGT GCAAAAGATA CGATAAAGAA |
| 321 | ACAAATATCT AAATATATAA ATGACTCAGA GAAATTTAAG |
| 361 | AATTTGTTTA ATCAAAACCT TATCGATGCT AAAAAAGGGC |
| 401 | AAGAGTCAGA TTTAATTCTA TGGCTAAAGC AATCTAAGGA |
| 441 | TAATGGCATA GAACTATTTA AAGCTAATAG TGATATCACA |
| 481 | GATATAGATG AGGCGTTAGA AATAATCAAA TCTTTTAAAG |
| 521 | GTTGGACAAC TTATTTTAAG GGTTTTCATG AAAATAGAAA |
| 561 | AAATGTTTAT AGTAGCAATG ATATTCCTAC ATCTATTATT |
| 601 | TATAGGATAG TAGATGATAA TTTGCCTAAA TTTCTAGAAA |
| 641 | ATAAAGCTAA GTATGAGAGT TTAAAAGACA AAGCTCCAGA |
| 681 | AGCTATAAAC TATGAACAAA TTAAAAAAGA TTTGGCAGAA |
| 721 | GAGCTAACCT TTGATATTGA CTACAAAACA TCTGAAGTTA |
| 761 | ATCAAAGAGT TTTTTCACTT GATGAAGTTT TTGAGATAGC |
| 801 | AAACTTTAAT AATTATCTAA ATCAAAGTGG TATTACTAAA |
| 841 | TTTAATACTA TTATTGGTGG TAAATTTGTA AATGGTGAAA |
| 881 | ATACAAAGAG AAAAGGTATA AATGAATATA TAAATCTATA |
| 921 | CTCACAGCAA ATAAATGATA AAACACTCAA AAAATATAAA |
| 961 | ATGAGTGTTT TATTTAAGCA AATTTTAAGT GATACAGAAT |
| 1001 | CTAAATCTTT TGTAATTGAT AAGTTAGAAG ATGATAGTGA |
| 1041 | TGTAGTTACA ACGATGCAAA GTTTTTATGA GCAAATAGCA |
| 1081 | GCTTTTAAAA CAGTAGAAGA AAAGTCTATT AAGGAAACAC |
| 1121 | TATCTTTACT ATTTGATGAT TTAAAAGCTC AAAAACTTGA |
| 1161 | TTTGAGTAAA ATTTATTTTA AAAATGATAA ATCTCTTACT |
| 1201 | GATCTATCAC AACAAGTTTT TGATGATTAT AGTGTTATTG |
| 1241 | GTACAGCGGT ACTAGAATAT ATAACTCAAC AAGTAGCACC |
| 1281 | TAAAAATCTT GATAACCCTA GTAAGAAAGA GCAAGATTTA |
| 1321 | ATAGCCAAAA AAACTGAAAA AGCAAAATAC TTATCTCTAG |
| 1361 | AAACTATAAA GCTTGCCTTA GAAGAATTTA ATAAGCATAG |
| 1401 | AGATATAGAT AAACAGTGTA GGTTTGAAGA AATACTTTCA |
| 1441 | AACTTTGCGG CTATTCCGAT GATATTTGAT GAAATAGCTC |
| 1481 | AAAACAAAGA CAATTTGGCA CAGATATCTA TCAAATACCA |
| 1521 | AAATCAAGGT AAAAAAGACC TACTTCAAGC TAGTGCAGAA |
| 1561 | GAAGATGTTA AAGCTATCAA GGATCTTTTA GATCAAACTA |
| 1601 | ATAATCTCTT GCATAGGCTA AAAATATTTC ATATTAGTCA |
| 1641 | ATCAGAAGAT AAGGCAAATA TTTTAGACAA GGATGAGCAT |
| 1681 | TTTTATCTAG TATTTGAGGA GTGCTACTTT GAGCTAGCGA |
| 1721 | ATATAGTGCC TCTTTATAAC AAAATTAGAA ACTATATAAC |
| 1761 | TCAAAAGCCA TATAGTGATG AGAAATTTAA GCTCAATTTT |
| 1801 | GAGAACTCGA CTTTGGCGAG TGGCTGGGAC AAAAACAAAG |
| 1841 | AGTCTGCTAA TACAGCAATT TTATTTATCA AAGATGATAA |
| 1881 | ATATTATCTT GGTATTATGG ATAAAAAACA TAACAAAATA |
| 1921 | TTTAGCGATA AAGCTATTGA AGAAAATAAA GGAGAAGGCT |
| 1961 | ACAAGAAAAT TGTTTATAAG CAAATTGCAG ATGCCTCAAA |
| 2001 | AGATATCCAA AATTTGATGA TTATTGATGG AAAAACTGTA |
| 2041 | TGTAAAAAAG GCAGAAAAGA TCGAAATGGA GTAAACAGGC |
| 2081 | AGTTACTAAG CTTAAAGAGA AAACATTTGC CAGAGAATAT |
| 2121 | ATATCGTATT AAAGAAACTA AAAGCTATTT AAAGAATGAA |
| 2161 | GCAAGATTTA GTAGAAAAGA TTTATATGAT TTTATAGATT |
| 2201 | ATTACAAAGA TAGACTTGAT TATTATGACT TTGAATTTGA |
| 2241 | GCTAAAGCCA TCAAATGAGT ATTCAGATTT TAATGATTTT |
| 2281 | ACTAACCATA TTGGTTCTCA AGGCTATAAA CTAACATTTG |
| 2321 | AGAATATATC TCAAGACTAT ATAAATAGTC TAGTAAATGA |
| 2361 | AGGCAAACTT TATTTGTTCC AAATCTATAG TAAAGATTTT |
| 2401 | TCAGCTTATA GCAAAGGGCG ACCAAATCTA CATACTTTAT |
| 2441 | ATTGGAAAGC GCTGTTTGAT GAGAGAAATC TTCAAGATGT |
| 2481 | GGTTTATAAG CTAAATGGTG AGGCAGAGCT TTTTTATCGT |
| 2521 | AAACAATCAA TACCTAAAAA AATCACTCAC CCAGCCAAAG |
| 2561 | AGACAATAGC TAATAAAAAC AAAGATAATC CTAAAAAAGA |
| 2601 | GAGTGTTTTT GAATATGATT TAATCAAGGA TAAACGCTTT |
| 2641 | ACTGAAGATA AGTTTTTCTT TCACTGTCCT ATTACAATCA |
| 2681 | ATTTTAAATC TAGTGGAGCT AATAAGTTTA ATGATGAAAT |
| 2721 | CAATTTATTG CTAAAAGAAA AAGCAAATGA TGTTCATATA |
| 2761 | TTAAGTATAG ATAGAGGTGA AAGACATTTA GCTTACTATA |
| 2801 | CTTTGGTAGA TGGTAAAGGA AATATTATCA AGCAAGATAA |
| 2841 | TTTCAACATC ATTGGTAACG ATAGAATGAA AACAAACTAC |
| 2881 | CATGATAAGC TTGCTGCAAT AGAGAAAGAT AGGGATTCAG |
| 2921 | CTAGGAAAGA CTGGAAAAAG ATAAATAACA TCAAAGAGAT |
| 2961 | GAAAGAGGGC TATCTATCTC AGGTAGTTCA TGAAATAGCT |
| 3001 | AAGCTAGTTA TAGAGTATAA TGCTATTGTG GTTTTTGAGG |
| 3041 | ATTTAAATTT TGGATTTAAA AGAGGGCGTT TCAAGGTAGA |
| 3081 | GAAGCAGGTC TATCAAAAGT TAGAAAAAAT GCTAATTGAG |
| 3121 | AAACTAAACT ATCTAGTTTT CAAAGATAAT GAGTTTGATA |
| 3161 | AAACTGGGGG AGTGCTTAGA GCTTATCAGC TAACAGCACC |
| 3201 | TTTTGAGACT TTTAAAAAGA TGGGTAAACA AACAGGTATT |
| 3241 | ATCTACTATG TACCAGCTGG TTTTACTTCA AAAATTTGTC |
| 3281 | CTGTAACTGG TTTTGTAAAT CAGTTATATC CTAAGTATGA |
| 3321 | AAGTGTCAGC AAATCTCAAG AGTTCTTTAG TAAGTTTGAC |
| 3361 | AAGATTTGTT ATAACCTTGA TAAGGGCTAT TTTGAGTTTA |
| 3401 | GTTTTGATTA TAAAAACTTT GGTGACAAGG CTGCCAAAGG |
| 3441 | CAAGTGGACT ATAGCTAGCT TTGGGAGTAG ATTGATTAAC |
| 3481 | TTTAGAAATT CAGATAAAAA TCATAATTGG GATACTCGAG |
| 3521 | AAGTTTATCC AACTAAAGAG TIGGAGAAAT TGCTAAAAGA |
| 3561 | TTATTCTATC GAATATGGGC ATGGCGAATG TATCAAAGCA |
| 3601 | GCTATTTGCG GTGAGAGCGA CAAAAAGTTT TTTGCTAAGC |
| 3641 | TAACTAGTGT CCTAAATACT ATCTTACAAA TGCGTAACTC |
| 3681 | AAAAACAGGT ACTGAGTTAG ATTATCTAAT TTCACCAGTA |
| 3721 | GCAGATGTAA ATGGCAATTT CTTTGATTCG CGACAGGCGC |
| 3761 | CAAAAAATAT GCCTCAAGAT GCTGATGCCA ATGGTGCTTA |
| 3801 | TCATATTGGG CTAAAAGGTC TGATGCTACT AGATAGGATC |
| 3841 | AAAAATAATC AAGAGGGCAA AAAACTCAAT TTGGTTATCA |
| 3881 | AAAATGAAGA GTATTTTGAG TTCGTGCAGA ATAGGAATAA |
| 3921 | CTAA |

Another example of a type V CRISPR-associated protein Cas12a/Cpf1 Francisella tularensis nuclease can have the following sequence (NCBI WP_003040289.1; SEQ ID NO:3).

| | |
|---|---|
| 1 | MSIYQEFVNK YSLSKTLRFE LIPQGKTLEN IKARGLILDD |
| 41 | EKRAKDYKKA KQIIDKYHQF FIEEILSSVC ISEDLLQNYS |
| 81 | DVYFKLKKSD DDNLQKDFKS AKDTIKKQIS EYIKDSEKFK |
| 121 | NLFNQNLIDA KKGQESDLIL WLKQSKDNGI ELFKANSDIT |
| 161 | DIDEALEIIK SFKGWTTYFK GFHENRKNVY SSNDIPTSII |
| 201 | YRIVDDNLPK FLENKAKYES LKDKAPEAIN YEQIKKDLAE |
| 241 | ELTFDIDYKT SEVNQRVFSL DEVFEIANFN NYLNQSGITK |
| 281 | FNTIIGGKFV NGENTKRKGI NEYINLYSQQ INDKTLKKYK |
| 321 | MSVLFKQILS DTESKSFVID KLEDDSDVVT TMQSFYEQIA |
| 361 | AFKTVEEKSI KETLSLLFDD LKAQKLDLSK IYFKNDKSLT |
| 401 | DLSQQVFDDY SVIGTAVLEY ITQQIAPKNL DNPSKKEQEL |
| 441 | IAKKTEKAKY LSLETIKLAL EEFNKHRDID KQCRFEEILA |
| 481 | NFAAIPMIFD EIAQNKDNLA QISIKYQNQG KKDLLQASAE |
| 521 | DDVKAIKDLL DQTNNLLHKL KIFHISQSED KANILDKDEH |
| 561 | FYLVFEECYF ELANIVPLYN KIRNYITQKP YSDEKFKLNF |
| 601 | ENSTLANGWD KNKEPDNTAI LFIKDDKYYL GVMNKKNNKI |
| 641 | FDDKAIKENK GEGYKKIVYK LLPGANKMLP KVFFSAKSIK |
| 681 | FYNPSEDILR IRNHSTHTKN GSPQKGYEKF EFNIEDCRKF |
| 721 | IDFYKQSISK HPEWKDFGFR FSDTQRYNSI DEFYREVENQ |
| 761 | GYKLTFENIS ESYIDSVVNQ GKLYLFQIYN KDFSAYSKGR |
| 801 | PNLHTLYWKA LFDERNLQDV VYKLNGEAEL FYRKQSIPKK |
| 841 | ITHPAKEAIA NKNKDNPKKE SVFEYDLIKD KRFTEDKFFF |
| 881 | HCPITINFKS SGANKFNDEI NLLLKEKAND VHILSIDRGE |
| 921 | RHLAYYTLVD GKGNIIKQDT FNIIGNDRMK TNYHDKLAAI |
| 961 | EKDRDSARKD WKKINNIKEM KEGYLSQVVH EIAKLVIEYN |
| 1001 | AIVVFEDLNF GFKRGRFKVE KQVYQKLEKM LIEKLNYLVF |
| 1041 | KDNEFDKTGG VLRAYQLTAP FETFKKMGKQ TGIIYYVPAG |
| 1081 | FTSKICPVTG FVNQLYPKYE SVSKSQEFFS KFDKICYNLD |
| 1121 | KGYFEFSFDY KNFGDKAAKG KWTIASFGSR LINFRNSDKN |
| 1161 | HNWDTREVYP TKELEKLLKD YSIEYGHGEC IKAAICGESD |
| 1201 | KKFFAKLTSV LNTILQMRNS KTGTELDYLI SPVADVNGNF |
| 1241 | FDSRQAPKNM PQDADANGAY HIGLKGLMLL GRIKNNQEGK |
| 1281 | KLNLVIKNEE YFEFVQNRNN |

The D917 and/or E1006 residues of the SEQ ID NO:3 protein (highlighted above) can be mutated to generate a catalytically inactive form or a form with reduce catalytic activity of this Cas12a (ddFnCas12a) protein.

In another example, a Francisella tularensis subsp. novicida U112 Cas12 nuclease, also referred to as an FnCas12a can have the following sequence (NCBI A0Q7Q2.1, SEQ ID NO:4).

| | |
|---|---|
| 1 | MSIYQEFVNK YSLSKTLRFE LIPQGKTLEN IKARGLILDD |
| 41 | EKRAKDYKKA KQIIDKYHQF FIEEILSSVC ISEDLLQNYS |
| 81 | DVYFKLKKSD DDNLQKDFKS AKDTIKKQIS EYIKDSEKFK |
| 121 | NLFNQNLIDA KKGQESDLIL WLKQSKDNGI ELFKANSDIT |
| 161 | DIDEALEIIK SFKGWTTYFK GFHENRKNVY SSNDIPTSII |
| 201 | YRIVDDNLPK FLENKAKYES LKDKAPEAIN YEQIKKDLAE |
| 241 | ELTFDIDYKT SEVNQRVFSL DEVFEIANFN NYLNQSGITK |
| 281 | FNTIIGGKFV NGENTKRKGI NEYINLYSQQ INDKTLKKYK |
| 321 | MSVLFKQILS DTESKSFVID KLEDDSDVVT TMQSFYEQIA |
| 361 | AFKTVEEKSI KETLSLLFDD LKAQKLDLSK IYFKNDKSLT |
| 401 | DLSQQVFDDY SVIGTAVLEY ITQQIAPKNL DNPSKKEQEL |
| 441 | IAKKTEKAKY LSLETIKLAL EEFNKHRDID KQCRFEEILA |
| 481 | NFAAIPMIFD EIAQNKDNLA QISIKYQNQG KKDLLQASAE |
| 521 | DDVKAIKDLL DQTNNLLHKL KIFHISQSED KANILDKDEH |
| 561 | FYLVFEECYF ELANIVPLYN KIRNYITQKP YSDEKFKLNF |
| 601 | ENSTLANGWD KNKEPDNTAI LFIKDDKYYL GVMNKKNNKI |
| 641 | FDDKAIKENK GEGYKKIVYK LLPGANKMLP KVFFSAKSIK |
| 681 | FYNPSEDILR IRNHSTHTKN GSPQKGYEKF EFNIEDCRKF |
| 721 | IDFYKQSISK HPEWKDFGFR FSDTQRYNSI DEFYREVENQ |
| 761 | GYKLTFENIS ESYIDSVVNQ GKLYLFQIYN KDFSAYSKGR |
| 801 | PNLHTLYWKA LFDERNLQDV VYKLNGEAEL FYRKQSIPKK |
| 841 | ITHPAKEAIA NKNKDNPKKE SVFEYDLIKD KRFTEDKFFF |
| 881 | HCPITINFKS SGANKFNDEI NLLLKEKAND VHILSIDRGE |
| 921 | RHLAYYTLVD GKGNIIKQDT FNIIGNDRMK TNYHDKLAAI |
| 961 | EKDRDSARKD WKKINNIKEM KEGYLSQVVH EIAKLVIEYN |
| 1001 | AIVVFEDLNF GFKRGRFKVE KQVYQKLEKM LIEKLNYLVF |
| 1041 | KDNEFDKTGG VLRAYQLTAP FETFKKMGKQ TGIIYYVPAG |
| 1081 | FTSKICPVTG FVNQLYPKYE SVSKSQEFFS KFDKICYNLD |
| 1121 | KGYFEFSFDY KNFGDKAAKG KWTIASFGSR LINFRNSDKN |
| 1161 | HNWDTREVYP TKELEKLLKD YSIEYGHGEC IKAAICGESD |
| 1201 | KKFFAKLTSV LNTILQMRNS KTGTELDYLI SPVADVNGNF |
| 1241 | FDSRQAPKNM PQDADANGAY HIGLKGLMLL GRIKNNQEGK |
| 1281 | KLNLVIKNEE YFEFVQNRNN |

The D917 and E1006 residues of the SEQ ID NO:4 protein (highlighted above) can be mutated to generate a catalytically inactive form or a form with reduce catalytic activity of this Cas12a (ddFnCas12a) protein.

Variants and homologs of these sequences can also be used in the methods and systems described herein. For example, such variants can have less than 100% sequence identity to any of the sequences described herein. The variants and homologs can have about at least 40% sequence identity, or at least 50% sequence identity, or at least 60% sequence identity, or at least 70% sequence identity, or at least 80% sequence identity, or at least 90% sequence identity, or at least 95% sequence identity, or at least 96% sequence identity, or at least 97% sequence identity, or at least 98% sequence identity, or at least 99% sequence identity, or 60-99% sequence identity, or 70-99% sequence identity, or 80-99% sequence identity, or 90-95% sequence identity, or 90-99% sequence identity, or 95-97% sequence identity, or 97-99% sequence identity, or 100% sequence identity with any of sequences described herein.

### Guide RNAs

A CRISPR guide RNA can be used that can target a Cas enzyme, including a defective Cas enzyme, to the desired location in the genome, where it can cleave the DNA for generation of a genomic modification.

A guide RNA interacts with CRISPR/Cas to guide it to a specific target site. Each guide RNA can comprise one or more regions, including for example, a first region that is complementary to the target site in the target sequence (targeting guide RNA (crRNA)) and a second region that forms one or more stem loop structures, the trans-activating CRISPR RNA/nuclease-binding guide RNA (tracrRNA).

The first region of each guide RNA is different such that each guide RNA guides CRISPR/Cas to a specific target site. The second region of each guide RNA can be the same in all guide RNAs.

The first region of the guide RNA is complementary to the target site in the target sequence such that the first region of the guide RNA can base pair with the target site. The guide RNAs can be designed to include one or more, for example, one or two targeting sequences. In various embodiments, the first region of the guide RNA can comprise from about 10 nucleotides to more than about 25 nucleotides. For example, the region of base pairing between the first region of the guide RNA and the target site in the target sequence can be about 10, 11, 12, 13, 14, 15, 16, 17, 18, 19, 20, 22, 23, 24, 25, or more than 25 nucleotides in length.

The guide RNA also comprises a second region that forms a secondary structure. In some embodiments, the secondary structure comprises one or more stem (or hairpin) and loop structures. The length of the loop and the stem can vary. For example, the loop can range from about 3 to about 10 nucleotides in length, and the stem can range from about 3 to about 20 base pairs in length. The stem can comprise one or more bulges of 1 to about 10 nucleotides. Thus, the overall length of the second region can range from about 10 to about 60 nucleotides in length. In some embodiments, the guide RNAs can have one, two, three or four stem structures with double-stranded regions of about 3-5 nucleotides.

The gRNA can have 1 or more spaces, such as 1, 2, 3, 4, or 5 spacers. In one embodiment, the gRNA has 2 spacers.

A variety of guide RNAs can be used to modify A. ferrooxidans. Cas12a nucleases can use guide RNAs that include both the targeting guide RNA (crRNA) sequences and the nuclease-binding guide RNA (tracrRNA) sequences. The guide RNAs for use with Cas12 nucleases also target sites near TTTV PAM sites.

In embodiments in which the guide RNA is introduced into the cell as a DNA molecule, the guide RNA coding sequence can be operably linked to promoter control sequence for expression of the guide RNA in the cell. For example, the RNA coding sequence can be operably linked to a promoter sequence that is recognized by polymerase.

The DNA molecule encoding the guide RNA can be linear or circular. In some embodiments, the DNA sequence encoding the guide RNA can be part of a vector. The vector can comprise additional expression control sequences, selectable marker sequences, origins of replication, and the like.

The invention will be further described by the following non-limiting examples.

### EXAMPLES

### Experimental Materials

Strains used in this study include E. coli DH10β and E. coli BL21 obtained from NEB (Ipswich, MA), E. coli S17-1 ATCC 47055 and A. ferrooxidans ATCC 23270 purchased from ATCC (Manassas, Virginia). All A. ferrooxidans strains were initially grown in 100 mL of iron and sulfur growth media (F2S medium) with an initial optical density measured at 600 nm (0D600) of 0.001, which corresponds to a cell density of 8.3×10⁶ cells/mL, in shaking incubator (30° C. and 140 rpm). The F2S medium consisted of (NH₄)₂SO₄, 0.8 g/L; HK₂PO₄, 0.1 g/L; MgSO₄·7H₂O, 2.0 g/L; Trace mineral solution (MD-TMS, ATCC), 5 mL/L; citric acid, 1.92 g/L; FeSO₄·7H₂O, 27.8 g/L; and dispersed sulfur (#S789400, Toronto Research Chemicals), 0.1% (w/v). The media was filtered through a 0.2 μm pore size (Thermo Fisher Scientific, Waltham, MA) prior to use, and sulfur was added to the media after filtration. The pFnCpf1_min (pY002) plasmid expressing FnCpf1 (Cas12a) and spacers 1-4 of CRISPR array following the J23119 promoter, from Francisella tularensis subsp. Novicida, was sourced from Addgene (#69975).

Enzymes and reagents for DNA manipulation were obtained from NEB, and oligonucleotides were purchased from Integrated DNA Technologies (Coralville, Iowa).

Given the 4 native gRNA sequences that are from the original F. novicida strain following the J23119 promoter, the sequences up to the end of the second first native gRNA (with direct repeat sequence of 5′ GTCTAAGAACTTTAAATAATTTCTACTGTTGTAGAT) were amplified and cloned into pYI11 vector, the empty pJRD vector with tac promoter, via NEBuilder HiFi DNA Assembly following the manufacturer's instructions. The resulting construct (pJRD_Cas12a) was then converted into a catalytically inactivated form by a series of E1006A and D917A double mutations were made on the pJRD_Cas12a via Q5 Site-Directed Mutagenesis Kit (NEB). The final plasmid (pJRD_dCas12a) was referred to as dCas12a.

### Example 1: Optimizing Guide RNAs for FnCas12a

A catalytically active FnCas12a was introduced into the pJRD (pJRD215 plasmid/vector; other vectors can include pBBR (Yamada et al. Journal of Bioscience and Bioengineering 133 (2) 105e109 (2022)); pBBR1-MCS2), a mobilizable IncQ cloning vector with a broad host range. The pJRD plasmid was introduced into A. ferrooxidans by conjugation (mating) with the plasmid-carrying E. coli, which is challenging as A. ferrooxidans thrive under iron-rich (≤100 mM Fe) and acidic (pH<2.0) conditions. Because conjugal transfer is a rare event between E. coli and A. ferrooxidans, growth and selective media formulations were developed and optimized to improve the environmental conditions of the two bacteria placed in suboptimal conditions and to increase the transfer frequencies. Other workers (e.g., Yamada et al. (2022)), used Peng mating media and 2:2 selection media (Table 1 in Jung et al., 2021, Trends in Biotechnol, doi.org/10.1016/j.tibtech.2021.10.004), whereas the experiments provided herein used the Inaba mating media and S204 selection media (Table 1 in Jung et al., 2021), which were modified from the initial formulations for better efficiency by substituting Fe²⁺ with Fe³⁺, including diaminopimelic acid and leucine, while adjusting concentrations of thiosulfate and other nutrients (ammonium, magnesium, and potassium chloride). The successful conjugation of the plasmid confirmed that ddFnCas12a did not have adverse effects on the cells.

It was important to control Cas gene expression but control over gene expression in A. ferrooxidans is challenging. The most commonly used promoter, tac, resulted in high levels of constitutive expression. Therefore, the FnCas12a was converted to a catalytically inactive form (ddFnCas12a) by introducing a D917A/E1006A double mutation. Such a double mutation of the FnCas12a protein can successfully be used for CRISPR interference (CRISPRi), allowing multiplex gene editing via autonomous processing of a single gRNA array.

Guide RNA (gRNA) sequences were generated for desired target sequences near TTTV PAM sites. In experiments, gRNAs were designed to evaluate whether one or two spacers, and whether various secondary structure could improve editing of an E. coli LacY gene, under M9-0.4% lactose. The gRNAs were transfected into E. coli BL21 cells expressing the ddFnCas12a enzyme, and cell growth was monitored in M9 media containing 0.4% lactose as the sole carbon source.

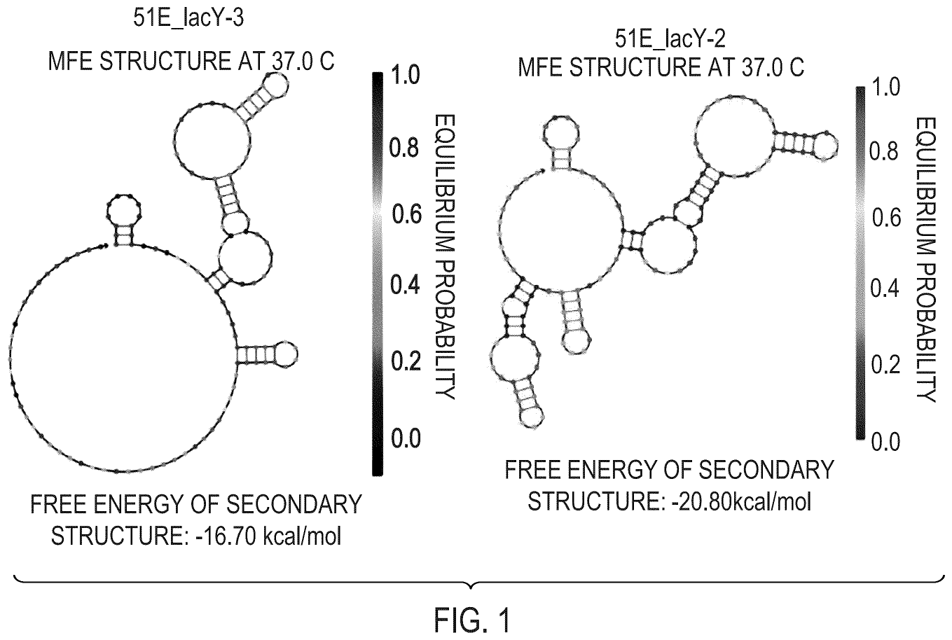

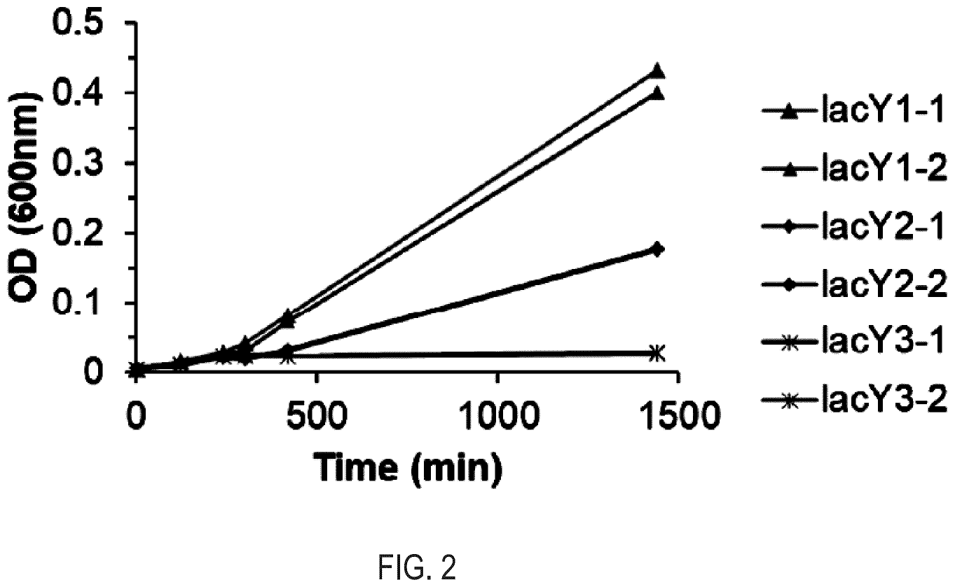

Structures of guide RNAs having one or two spacers, and various secondary structures are shown in FIG. 1. When 1-spacer was targeted, the improved secondary RNA structures with visible folding of direct repeats (lacY2-1 and 2-2) had more impact on growth suppression than with additional secondary RNA structures (lacY1-1 and 1-2). Furthermore, having 2-spacers with minimized secondary gRNA structure (lacY3-1 and 3-2) completely suppressed the cell growth, indicating that having 2 spacers targeted to a single gene increases the possibility that a good gRNA sequence is selected, will edit a target site, especially when the secondary gRNA structure is minimized (FIGS. 1 and 2).

### Example 2: Editing of Acidithiobacillus ferrooxidans

Using the methods and systems designed as indicated in Example 1, an endogenous Acidithiobacillus ferrooxidans gene was edited/knocked down by ddFnCas12a (introducing ddFnCas12a with a gRNA which targets petBII). While A. ferrooxidans can derive energy from oxidation of both iron and sulfur, iron oxidation is generally inhibited under sulfur-rich conditions. To address this problem, gRNAs were designed to target petBII, an enzyme that has a key role in sulfur oxidation of A. ferrooxidans.

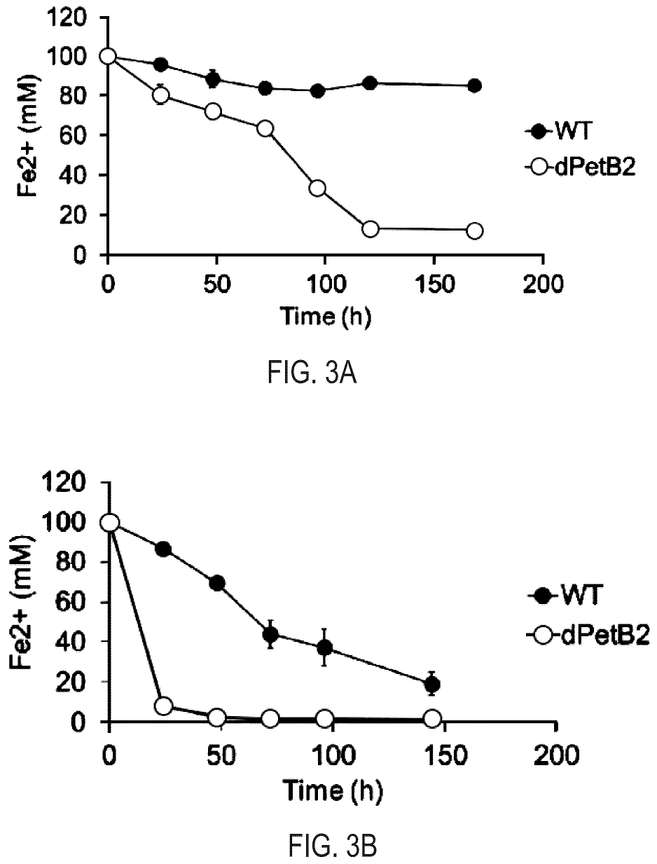

As shown in FIG. 3A, when petBII expression was knocked down, which has a key role in sulfur oxidation of A. ferrooxidans, the A. ferrooxidans knockdown cells overcame repressed iron oxidation that occurred under high sulfur conditions (5 g S/L) in wild-type A. ferrooxidans cells. Faster iron oxidation was also observed in the knock down A. ferrooxidans cells under low sulfur conditions (1 g S/L) (FIG. 3B).

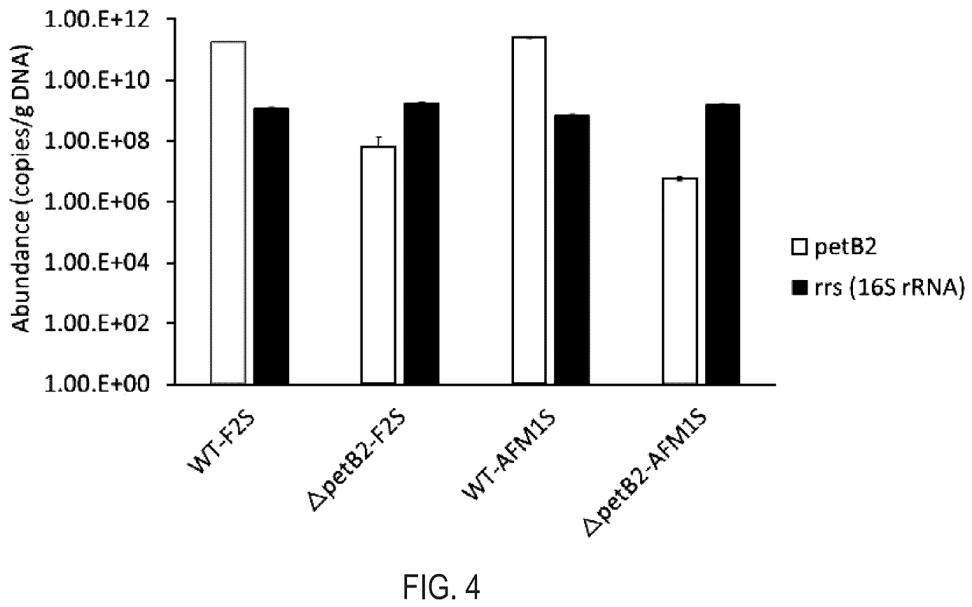

Much lower transcriptional expression of petBII, in relation to 16S rRNA gene, was observed in the knock down A. ferrooxidans cells under iron and sulfur media, which demonstrated the successful knock down efficacy using the ddFnCas12a/guide RNA system (FIG. 4).

The editing systems and methods described herein therefore provide successful CRISPR/Cas interference technologies for editing A. ferrooxidans. The editing systems and methods described herein can be used for manipulation of gene expression in vivo, thereby providing useful modifications of A. ferrooxidans cellular metabolism.

### BIBLIOGRAPHY

1. Schippers A, Hedrich S, Vasters J, Drobe M, Sand W, Willscher S. Biomining: metal recovery from ores with microorganisms. Adv Biochem Eng Biotechnol. 2014; 141: pp. 1-47.
2. Valdés J, Pedroso I, Quatrini R, Dodson R J, Tettelin H, Blake R 2nd, Eisen J A, Holmes DS. Acidithiobacillus ferrooxidans metabolism: from genome sequence to industrial applications. BMC Genomics. 2008 Dec. 11; 9:597.
3. Jung H, Inaba Y, Banta S. Genetic engineering of the acidophilic chemolithoautotroph Acidithiobacillus ferrooxidans. Trends Biotechnol. 2021 Nov. (21).
4. Yamada S, Suzuki Y, Kouzuma A, Watanabe K. Development of a CRISPR interference system for selective gene knockdown in Acidithiobacillus ferrooxidans. J Biosci Bioeng. 2022 February; 133(2): pp. 105-109.
5. Liu Z, Dong H, Cui Y, Cong L, Zhang D. Application of different types of CRISPR/Cas-based systems in bacteria. Microb Cell Fact. 2020 Sep. 3; 19(1):172.

All patents and publications referenced or mentioned herein are indicative of the levels of skill of those skilled in the art to which the invention pertains.

All publications, patents and patent applications are incorporated herein by reference. While in the foregoing specification, this invention has been described in relation to certain preferred embodiments thereof, and many details have been set forth for purposes of illustration, it will be apparent to those skilled in the art that the invention is susceptible to additional embodiments and that certain of the details herein may be varied considerably without departing from the basic principles of the invention.

The specific compositions and methods described herein are representative, exemplary and not intended as limitations on the scope of the invention. Other objects, aspects, and embodiments will occur to those skilled in the art upon consideration of this specification and are encompassed within the spirit of the invention as defined by the scope of the claims. It will be readily apparent to one skilled in the art that varying substitutions and modifications may be made to the invention disclosed herein without departing from the scope and spirit of the invention. The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by embodiments and optional features, modification and variation of the concepts herein disclosed may be resorted to by those skilled in the art, and that such modifications and variations are considered to be within the scope of this invention as defined by the appended claims and statements of the invention.

The invention illustratively described herein may be practiced in the absence of any element or elements, or limitation or limitations, which is not specifically disclosed herein as essential. The methods and processes illustratively described herein may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a guide RNA” or “a nuclease” or “a cell” includes a plurality of such guide RNAs, nucleases or cells, and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

The Abstract is provided to comply with 37 C.F.R. § 1.72(b) to allow the reader to quickly ascertain the nature and gist of the technical disclosure. The Abstract is submitted with the understanding that it will not be used to interpret or limit the scope or meaning of the claims.

What is claimed is:

1. A method comprising introducing at least one guide RNA to at least one Acidithiobacillus ferrooxidans cell that expresses a Cas12 nuclease or modified Cas12 nuclease, to generate at least one modified Acidithiobacillus ferrooxidans cell.

2. The method of claim 1, wherein the Cas12 nuclease or the modified Cas12 nuclease is a Cas12a nuclease or a modified Cas12a nuclease.

3. The method of claim 1, wherein the Cas12 nuclease or the modified Cas12 nuclease is a Francisella tularensis Cas12a nuclease or a modified Francisella tularensis Cas12a nuclease.

4. The method of claim 1, wherein the modified Cas12 nuclease has reduced catalytic activity as compared to a wild type Cas12 nuclease, wherein the modified Cas12 nuclease can perform genomic editing.

5. The method of claim 1, wherein the modified Cas12 nuclease is a catalytically inactive form of Cas12a that can perform genomic editing.

6. The method of claim 1, wherein the modified Cas12 nuclease has a mutation at D917 of SEQ ID NO: 1, 3 or 4 or 95% identity thereto.

7. The method of claim 1, wherein the mutation is D917A.

8. The method of claim 1, where in the modified Cas12 nuclease has a mutation at E1005 of SEQ ID NO: 1, E1006 of SEQ ID NO: 3, E1006 of SEQ ID NO: 4 or 95% identity thereto.

9. The method of claim 1, wherein the mutation is E1005A or E1006A.

10. The method of claim 1, wherein the modified Cas12 nuclease has a D917A mutation and an E1006A or E1005A mutation.

11. The method of claim 1, wherein at least one of the guide RNAs comprises one or more targeting sequences.

12. The method of claim 1, wherein at least one of the guide RNAs comprises two or more targeting sequences.

13. The method of claim 1, wherein at least one of the guide RNAs include both targeting guide RNA (crRNA) sequences and nuclease-binding guide RNA (tracrRNA) sequences in a single RNA molecule.

14. The method of claim 1, wherein at least one of the guide RNAs target genomic sites near TTTV PAM sites in the Acidithiobacillus ferrooxidans genome.

15. The method of claim 1, wherein at least one of the guide RNAs comprises up to two, three or four stem structures, each comprising double-stranded regions of about 3-5 nucleotides.

16. The method of claim 1, wherein at least one of the guide RNAs comprises one or two spacers.

17. The method of claim 1, wherein at least one of the guide RNAs are introduced to at least one Acidithiobacillus ferrooxidans cell as an expression cassette or expression vector comprising a promoter operably linked to a segment encoding at least one of the guide RNAs.

18. A system comprising (a) an expression cassette or expression vector comprising a promoter operably linked to restriction site adapted to receive a nucleotide segment encoding at least one guide RNA; and (b) a population of Acidithiobacillus ferrooxidans cells modified to express a Cas12 nuclease or a modified Cas12 nuclease.

19. The system of claim 18, wherein the Cas12 nuclease or the modified Cas12 nuclease is a Cas12a nuclease or a modified Cas12a nuclease.

20. The system of claim 18, wherein the Cas12 nuclease or the modified Cas12 nuclease is a Francisella tularensis Cas12a nuclease or a modified Francisella tularensis Cas12a nuclease.

21. The system of claim 18, wherein the modified Cas12 nuclease has reduced catalytic activity as compared to a wild type Cas12 nuclease, wherein the modified Cas12 nuclease can perform genomic editing.

22. The system of claim 18, wherein the modified Cas12 nuclease is a catalytically inactive form of Cas12a that can perform genomic editing.

23. The system of claim 18, wherein the modified Cas12 nuclease has a mutation at D917 of SEQ ID NO: 1, 3 or 4 or 95% identity thereto.

24. The system of claim 18, wherein the mutation is D917A.

25. The system of claim 18, where in the modified Cas12 nuclease has a mutation at E1005 of SEQ ID NO: 1, E1006 of SEQ ID NO: 3, E1006 of SEQ ID NO: 4 or 95% identity thereto.

26. The system of claim 18, wherein the mutation is E1005A or E1006A.

27. The system of claim 18, wherein the modified Cas12 nuclease has a D917A mutation and an E1006A or E1005A mutation.

28. The system of claim 18, wherein at least one of the guide RNAs comprises one or more targeting sequences.

29. The system of claim 18, wherein at least one of the guide RNAs comprises two or more targeting sequences.

30. The system of claim 18, wherein at least one of the guide RNAs include both targeting guide RNA (crRNA) sequences and nuclease-binding guide RNA (tracrRNA) sequences in a single RNA molecule.

31. The system of claim 18, wherein at least one of the guide RNAs target genomic sites near TTTV PAM sites in the Acidithiobacillus ferrooxidans genome.

32. The system of claim 18, wherein at least one of the guide RNAs comprises up to two, three or four stem structures, each comprising double-stranded regions of about 3-5 nucleotides.

33. The system of claim 18, wherein at least one of the guide RNAs comprises one or two spacers.


SEQUENCE LISTING (4 sequences)

SEQ ID NO: 1 - protein, 1307 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISKYINDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQVAPKNLDNPSKKEQDLIAKKTEKAKYLSLETIKLALEEFNKHRDIDKQCRFEEILSNFAAIPMIFDEIAQNKDNLAQISIKYQNQGKKDLLQASAEEDVKAIKDLLDQTNNLLHRLKIFHISQSEDKANILDKDEHFYLVFEECYFELANIVPLYNKIRNYITQKPYSDEKFKLNFENSTLASGWDKNKESANTAILFIKDDKYYLGIMDKKHNKIFSDKAIEENKGEGYKKIVYKQIADASKDIQNLMIIDGKTVCKKGRKDRNGVNRQLLSLKRKHLPENIYRIKETKSYLKNEARFSRKDLYDFIDYYKDRLDYYDFEFELKPSNEYSDFNDFTNHIGSQGYKLTFENISQDYINSLVNEGKLYLFQIYSKDFSAYSKGRPNLHTLYWKALFDERNLQDVVYKLNGEAELFYRKQSIPKKITHPAKETIANKNKDNPKKESVFEYDLIKDKRFTEDKFFFHCPITINFKSSGANKFNDEINLLLKEKANDVHILSIDRGERHLAYYTLVDGKGNIIKQDNFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLDRIKNNQEGKKLNLVIKNEEYFEFVQNRNN

SEQ ID NO: 2 - DNA, 3924 bp, Francisella tularensis
  source 1..3924: mol_type = other DNA
atgtcaatttatcaagaatttgttaataaatatagtttaagtaaaactctaagatttgagttaatcccacagggtaaaacacttgaaaacataaaagcaagaggtttgattttagatgatgagaaaagagctaaagactacaaaaaggctaaacaaataattgataaatatcatcagttttttatagaggagatattaagttcggtttgtattagcgaagatttattacaaaactattctgatgtttattttaaacttaaaaagagtgatgatgataatctacaaaaagattttaaaagtgcaaaagatacgataaagaaacaaatatctaaatatataaatgactcagagaaatttaagaatttgtttaatcaaaaccttatcgatgctaaaaaagggcaagagtcagatttaattctatggctaaagcaatctaaggataatggcatagaactatttaaagctaatagtgatatcacagatatagatgaggcgttagaaataatcaaatcttttaaaggttggacaacttattttaagggttttcatgaaaatagaaaaaatgtttatagtagcaatgatattcctacatctattatttataggatagtagatgataatttgcctaaatttctagaaaataaagctaagtatgagagtttaaaagacaaagctccagaagctataaactatgaacaaattaaaaaagatttggcagaagagctaacctttgatattgactacaaaacatctgaagttaatcaaagagttttttcacttgatgaagtttttgagatagcaaactttaataattatctaaatcaaagtggtattactaaatttaatactattattggtggtaaatttgtaaatggtgaaaatacaaagagaaaaggtataaatgaatatataaatctatactcacagcaaataaatgataaaacactcaaaaaatataaaatgagtgttttatttaagcaaattttaagtgatacagaatctaaatcttttgtaattgataagttagaagatgatagtgatgtagttacaacgatgcaaagtttttatgagcaaatagcagcttttaaaacagtagaagaaaagtctattaaggaaacactatctttactatttgatgatttaaaagctcaaaaacttgatttgagtaaaatttattttaaaaatgataaatctcttactgatctatcacaacaagtttttgatgattatagtgttattggtacagcggtactagaatatataactcaacaagtagcacctaaaaatcttgataaccctagtaagaaagagcaagatttaatagccaaaaaaactgaaaaagcaaaatacttatctctagaaactataaagcttgccttagaagaatttaataagcatagagatatagataaacagtgtaggtttgaagaaatactttcaaactttgcggctattccgatgatatttgatgaaatagctcaaaacaaagacaatttggcacagatatctatcaaataccaaaatcaaggtaaaaaagacctacttcaagctagtgcagaagaagatgttaaagctatcaaggatcttttagatcaaactaataatctcttgcataggctaaaaatatttcatattagtcaatcagaagataaggcaaatattttagacaaggatgagcatttttatctagtatttgaggagtgctactttgagctagcgaatatagtgcctctttataacaaaattagaaactatataactcaaaagccatatagtgatgagaaatttaagctcaattttgagaactcgactttggcgagtggctgggacaaaaacaaagagtctgctaatacagcaattttatttatcaaagatgataaatattatcttggtattatggataaaaaacataacaaaatatttagcgataaagctattgaagaaaataaaggagaaggctacaagaaaattgtttataagcaaattgcagatgcctcaaaagatatccaaaatttgatgattattgatggaaaaactgtatgtaaaaaaggcagaaaagatcgaaatggagtaaacaggcagttactaagcttaaagagaaaacatttgccagagaatatatatcgtattaaagaaactaaaagctatttaaagaatgaagcaagatttagtagaaaagatttatatgattttatagattattacaaagatagacttgattattatgactttgaatttgagctaaagccatcaaatgagtattcagattttaatgattttactaaccatattggttctcaaggctataaactaacatttgagaatatatctcaagactatataaatagtctagtaaatgaaggcaaactttatttgttccaaatctatagtaaagatttttcagcttatagcaaagggcgaccaaatctacatactttatattggaaagcgctgtttgatgagagaaatcttcaagatgtggtttataagctaaatggtgaggcagagcttttttatcgtaaacaatcaatacctaaaaaaatcactcacccagccaaagagacaatagctaataaaaacaaagataatcctaaaaaagagagtgtttttgaatatgatttaatcaaggataaacgctttactgaagataagtttttctttcactgtcctattacaatcaattttaaatctagtggagctaataagtttaatgatgaaatcaatttattgctaaaagaaaaagcaaatgatgttcatatattaagtatagatagaggtgaaagacatttagcttactatactttggtagatggtaaaggaaatattatcaagcaagataatttcaacatcattggtaacgatagaatgaaaacaaactaccatgataagcttgctgcaatagagaaagatagggattcagctaggaaagactggaaaaagataaataacatcaaagagatgaaagagggctatctatctcaggtagttcatgaaatagctaagctagttatagagtataatgctattgtggtttttgaggatttaaattttggatttaaaagagggcgtttcaaggtagagaagcaggtctatcaaaagttagaaaaaatgctaattgagaaactaaactatctagttttcaaagataatgagtttgataaaactgggggagtgcttagagcttatcagctaacagcaccttttgagacttttaaaaagatgggtaaacaaacaggtattatctactatgtaccagctggttttacttcaaaaatttgtcctgtaactggttttgtaaatcagttatatcctaagtatgaaagtgtcagcaaatctcaagagttctttagtaagtttgacaagatttgttataaccttgataagggctattttgagtttagttttgattataaaaactttggtgacaaggctgccaaaggcaagtggactatagctagctttgggagtagattgattaactttagaaattcagataaaaatcataattgggatactcgagaagtttatccaactaaagagttggagaaattgctaaaagattattctatcgaatatgggcatggcgaatgtatcaaagcagctatttgcggtgagagcgacaaaaagttttttgctaagctaactagtgtcctaaatactatcttacaaatgcgtaactcaaaaacaggtactgagttagattatctaatttcaccagtagcagatgtaaatggcaatttctttgattcgcgacaggcgccaaaaaatatgcctcaagatgctgatgccaatggtgcttatcatattgggctaaaaggtctgatgctactagataggatcaaaaataatcaagagggcaaaaaactcaatttggttatcaaaaatgaagagtattttgagttcgtgcagaataggaataactaa

SEQ ID NO: 3 - protein, 1300 aa, Francisella tularensis
MSIYQEFVNKYSLSKTLRFELIPQGKTLENIKARGLILDDEKRAKDYKKAKQIIDKYHQFFIEEILSSVCISEDLLQNYSDVYFKLKKSDDDNLQKDFKSAKDTIKKQISEYIKDSEKFKNLFNQNLIDAKKGQESDLILWLKQSKDNGIELFKANSDITDIDEALEIIKSFKGWTTYFKGFHENRKNVYSSNDIPTSIIYRIVDDNLPKFLENKAKYESLKDKAPEAINYEQIKKDLAEELTFDIDYKTSEVNQRVFSLDEVFEIANFNNYLNQSGITKFNTIIGGKFVNGENTKRKGINEYINLYSQQINDKTLKKYKMSVLFKQILSDTESKSFVIDKLEDDSDVVTTMQSFYEQIAAFKTVEEKSIKETLSLLFDDLKAQKLDLSKIYFKNDKSLTDLSQQVFDDYSVIGTAVLEYITQQIAPKNLDNPSKKEQELIAKKTEKAKYLSLETIKLALEEFNKHRDIDKQCRFEEILANFAAIPMIFDEIAQNKDNLAQISIKYQNQGKKDLLQASAEDDVKAIKDLLDQTNNLLHKLKIFHISQSEDKANILDKDEHFYLVFEECYFELANIVPLYNKIRNYITQKPYSDEKFKLNFENSTLANGWDKNKEPDNTAILFIKDDKYYLGVMNKKNNKIFDDKAIKENKGEGYKKIVYKLLPGANKMLPKVFFSAKSIKFYNPSEDILRIRNHSTHTKNGSPQKGYEKFEFNIEDCRKFIDFYKQSISKHPEWKDFGFRFSDTQRYNSIDEFYREVENQGYKLTFENISESYIDSVVNQGKLYLFQIYNKDFSAYSKGRPNLHTLYWKALFDERNLQDVVYKLNGEAELFYRKQSIPKKITHPAKEAIANKNKDNPKKESVFEYDLIKDKRFTEDKFFFHCPITINFKSSGANKFNDEINLLLKEKANDVHILSIDRGERHLAYYTLVDGKGNIIKQDTFNIIGNDRMKTNYHDKLAAIEKDRDSARKDWKKINNIKEMKEGYLSQVVHEIAKLVIEYNAIVVFEDLNFGFKRGRFKVEKQVYQKLEKMLIEKLNYLVFKDNEFDKTGGVLRAYQLTAPFETFKKMGKQTGIIYYVPAGFTSKICPVTGFVNQLYPKYESVSKSQEFFSKFDKICYNLDKGYFEFSFDYKNFGDKAAKGKWTIASFGSRLINFRNSDKNHNWDTREVYPTKELEKLLKDYSIEYGHGECIKAAICGESDKKFFAKLTSVLNTILQMRNSKTGTELDYLISPVADVNGNFFDSRQAPKNMPQDADANGAYHIGLKGLMLLGRIKNNQEGKKLNLVIKNEEYFEFVQNRNN

SEQ ID NO: 4 - protein, 1300 aa, Francisella tularensis; identical to SEQ ID NO: 3